(19) United States
(12) Patent Application Publication
(10) Pub. No.: US 2022/0068289 A1
(43) Pub. Date: Mar. 3, 2022

(54) Speech Processing Method and System in A Cochlear Implant

(71) Applicant: AidiSciTech Resarch Institute Co.,Ltd., Nanjing (CN)

(72) Inventors: Norden Eh HUANG, Nanjing (CN); Feng Qing Yang Zeng, Nanjing (CN)

(73) Assignee: AidiSciTech Resarch Institute Co.,Ltd., Nanjing (CN)

(21) Appl. No.: 17/357,982

(22) Filed: Jun. 25, 2021

(30) Foreign Application Priority Data
Sep. 3, 2020 (CN) 202010913039.8

(51) Int. Cl. G10L 21/0208; G10L 25/51; A61N 1/05

(52) U.S. Cl. CPC G10L 21/0208; G10L 2021/02087; A61N 1/0541; G10L 25/51

(57) ABSTRACT

The invention discloses a speech processing method and system in a cochlear implant. The method includes: obtaining a sound signal, and converting the sound signal into a digital signal; decomposing the digital signal using a mode decomposition method, obtaining a plurality of intrinsic mode functions, and converting the plurality of intrinsic mode functions into instantaneous frequencies and instantaneous amplitudes or instantaneous energy intensities; sorting the instantaneous frequencies to corresponding the preset electrode frequency bands of the electrodes in the cochlear implant; selecting N most energetic components from the corresponding frequency bands of the electrodes, and generating corresponding electrode stimulation signals according to the selected components. The present invention analyzes sound and composes the final electrode signals all in the time domain based on Hilbert-Huang transform; it is not limited by the principle of uncertainty, and there is no noise generated by harmonics.

### TECHINCAL FIELD

The invention relates to the field of cochlear implants, in particular to a speech processing method and system in a cochlear implant.

### BACKGROUND

Unlike hearing aids, which selectively amplify sound, the Cochlear Implants (CI) must create a sound by transmitting the sound signals directly to the afferent auditory nerves in the ears and then to the Primary Auditory Cortex (PAC). Therefore, CIs create the sensation of the sound directly for the Primary Auditory Cortex. In this sense, Cochlear Implant is a cure, not just a prosthetic, for severe hearing loss or even total deafness due to damage or defects of the middle and inner ears. It bypasses damaged portions of the ear to deliver processed sound signals to the auditory nerve in the cochlear directly. The current CIs are all based on the flawed assumption that the cochlear is a biological Fourier analyzer or a Fourier based Filter Bank. To overcome the flaws of the current cochlear implant design, the present inventive method is based on the adaptive Empirical Mode Decomposition method that works directly in the time domain, which is suitable for both nonlinear and nonstationary data without the limitation of uncertainty principle. It treats the cochlea as an EMD based Filter Bank, which offers a solution to most of the challenges face currently.

In a broader sense, the term, Cochlear Implant, used here also includes Brain Stem Implant and Bone Conduction Hearing Implant.

(1) Hearing Mechanism
In normal ear, acoustical signal is perceived as sound when the pressure wave associated with the acoustical signal propagates through the external auditory canal impinges on the tympanic membrane. This vibration is amplified through the ossicle mechanism (including the Malleus, Incus and Staple) to the oval window at the base of the cochlea. The vibration at the oval window then generates a traveling pressure wave in the vestibule, which will deform the soft Basilar membrane together with the organ of Corti and the stereocilia (or the hair cells) that touch the tectorial membrane and bend the hair cells. The bending of the hair cells at the peak of the wave will trigger the neurons to emit electric impulses that will travel through the thalamocortical system and be processed at the Primary Auditory Cortex (PAC) to produce the perceived sound.

(2) Hearing Loss
Hearing loss can result from any part of this long chain of events described above. If there is any dysfunction in the middle and inner ear that prevents the generation and transmission of the neural impulse to reach the PAC, we have a case of sensorineural hearing loss. Some of the hearing loss could be alleviated by non-invasive hearing aids, as in the case of hearing impairments due to ageing (presbycusis), over exposure to noise (Noise Induce of Hearing Loss, NIHL), heredity (congenital hearing loss), toxin from medications, and many others. However, hearing aids are completely useless for central deafness. For severe or total deaf patients, due to the absence of inner hair cells (IHC), Cochlear Implants would provide help, which are designed to substitute the function of the IHC by delivering the electric impulses from auditory stimuli directly to the thalamocortical system. For severe hearing impairment of total deafness, CIs would offer effective treatments.

Over the last three decades Cochlear Implants began to gain wide acceptance. Even though their performance is, in general, modest according to recent reviews by McDermott (2004) and Roche and Hansen (2015), the sound delivered by the implants however could relieve the total isolation of the patients and greatly improve their social function and life quality.

(3) The Principles of Cochlear Implants
The design principle of Cochlear Implants is fundamentally different from hearing aids. Hearing aids are based on the amplification of sound, more specifically the selective amplification of sound. The components of sound stimulation are modified and summed before the sound is produced and delivered to the ears as the single final sound. To preserve fidelity, the necessary condition requires only completeness of the components to produce the final sound.

Cochlear Implants are substitutes for the inner hair cells in the cochlea. The components are required to produce the proper electric stimuli at the electrodes planting at the proper location on the cochlea, where the final sensation of sound in the PAC is the sum of all the stimuli components. It requires each component making physical sense in additional of completeness of the sum of the components. However, due to the limited length of the implants, CI could not substitute the function of the 3500 inner hair cells completely. As a result, CI is a rather poor substitute for the cochlea; it lacks the fine frequency resolution provided by the 3500 nature inner hair cells. Indeed, all Cochlear Implants failed miserably for concurrent sound sources and especially the music sounds.

The essential components of the current CI system consist of microphone, speech processing unit including software and circuitry, induction coil pairs with stimulator and receiver, and electrodes. The basic principle of Cochlear Implants is as follows: The sound signals are first captured by a microphone, which are processed to extract some essential parameters before delivered to the implanted receiver through induction coil as electric signals. Then the electric signals are transmitted through the electrode array to the spiral ganglion neurons in the cochlea, which are transduced into local action potentials and delivered to the primary auditory cortex.

But the core of CIs is the proper selection of the frequency bands at any given moment, which is what this invention proposed to accomplish. Before discussing the principle of electrode selection, we will first discuss the problems of the present CI design.

(4) The Problems of the Current Cochlear Implant Design
The root of the problems of the current Cochlear Implant design is mis-conception upon which the sound perception was based on Fourier analysis. Ever since Helmholtz made the famous statement: “All sounds, no matter how complex, can be mathematically broken down into constituent sine waves, whether you know it or not,” sounds have been represented by Fourier frequency. This is far from the truth. Though both the acoustic and auditory communities study sounds, they seem to deal with different subjects. The acoustic community treats sound signals as physical entities, and uses frequency as standard to measure sounds. However, due to some seemingly anomalous phenomena, the auditory community finds some flaws in Fourier analysis, and treats sounds as sensations perceived by the brain through the mechanism of the ear, and uses pitch to quantify sounds, which unfortunately could not be measured objectively. Yet, most auditory experiments are still expressed in terms of frequency. This lands the neurobiological auditory study of sounds in a quandary. It is well known that to make sense of sounds, we need to perceive both the frequency of the carrier waves and their modulating waves (aka envelopes), a request beyond the capability of Fourier analysis (Huang et al 2016).

Based on the original discoverer of the function of cochlea, von Békésy (1974), the Basilar membrane movement, in response to the sounds, is a traveling wave. Its mechanism is determined by hydrodynamic principle. In fact, von Békésy stated clearly that “the application of Fourier analysis to hearing problems became more and more a handicap for research in hearing.” Most recently, Kim et al (2018, SPIE) and Motallebzadeh et al (2018, PNAS) modelled the basilar membrane with the organ of Corti based on the hydrodynamic principle and checked out its function perfectly.

Unfortunately, for Cochlear Implant systems, the sound signal processing has still been based on Fourier spectral analysis exclusively.

Cochlear Implants are intended to replace the function of the inner hair cells (about 3500 independent inner hair cells) with limited number of electrodes. However, there are severe limitations: First, only limited number of electrodes, around 25 is the maximum number; but to avoid cross talk, only 6 electrodes could be activated at the same time. Second, the implants can only cover 1¼, rather than entire three, circles of the cochlea, i.e., only about 40% of the total length near the basal end, but will contact 60% of the spiral ganglion cells that gives a ‘squeaky’ rat-like sounds. Third, the sound components from each electrode are rectified at the neural level; there is no chance of cancellation or combination among different components.

However, based on Smith et al (2002), the recognition of speech could be accomplished on the envelope of the sound components. Shannon et al (1995) demonstrate that suitably selected 4 components could be sufficient for language recognition. As a result, experience has indicated that the fewer components, the better, which is also in line with the sparse principle. Fourier components certainly would not meet this requirement, for the required components of a Fourier expansion is N/2, with N as the total data points. In fact, the more electrodes should be the better, for they would give better frequency differentiation. More electrodes, however, would cause ‘cross talk’ among different channels with no significant improvement in the performance. Various sound processing approaches had also been tried, such as Simultaneous Analog Signal (SAS); Compressive Analysis (CA); Continuous Interleaved Sampling (CIS); HiRes (High Resolution) devices; Advanced Combinatorial Encoders (ACE); Dynamic Peak Picking; Spectral Peak (SPEAK); and Current Steering. New processing methods notwithstanding, none of the available algorithms is clearly superior to any other, for most of them are Fourier based.

All the problems are due to the flawed Fourier Filter Bank assumption as summarized by McDermott (2004) and Schnupp et al (2011):
(1) In general, implant users can understand speech in quite condition with some training; but the pitch perception is in general poor. Auditory training programs would help;
(2) On average, implant users perceive rhythm in music nearly as well as listeners with normal hearing; but recognition of melodies is poor, with performance at little better than chance levels for many implant users;
(3) Perception of timbre is generally unsatisfactory; implant users tend to rate the quality of musical sounds near cacophony as less pleasant than listeners with normal hearing;
(4) For implant users who have usable acoustic hearing, at least for low-frequency sounds, perception of music is likely to be much better with combined acoustic and electric stimulation.

The problems are deeply rooted on the misconception of the audible sound theory as discussed in Huang and Yeh (2019). Although the auditory community collectively accept pitch as the putative standard for quantifying sounds, all experiments however are all based in terms of Fourier frequency, including Cochlear Implants. Fourier analysis is based on linear and stationary assumptions, but speech is neither linear nor stationary. For nonlinear signals, Fourier analysis representation would require artificial harmonics, which would lead to many problems such as missing fundamental.

In case of Cochlear Implants, the harmonics could cause additional problems. In CI, the electrode would only deliver electric stimuli proportional to the rectified frequency components. Thus, artificial harmonics loss the chance to combine and cancel each other, but are treated as real sound signals. As a result, the sums would result as unwanted noises. This is the reason why the less the number of electrodes actually the better the sound quality of CI as discussed above.

In this invention, we will present a new approach based on Empirical Mode Decomposition (EMD), which is designed for nonlinear and nonstationary signals, with a sparse representation, which is ideal for Cochlear Implants.

### BRIEF SUMMARY OF THE INVENTION

The object of the present invention is to provide a cochlear implant speech processing method and system, which is based on Empirical Mode Decomposition (EMD) or Hilbert-Huang Transform (HHT). In the present invention, it can provide the instantaneous frequency and the instantaneous energy of the sound at any given time, which is used to perform precise temporal analysis on the sound signal. By adopting the cochlear implant speech processing method and system of the present invention, the performance is good even when there are multiple sound sources at the same time, and music appreciation can even be performed.

The present invention is based on Empirical Mode Decomposition (EMD) defined sparse Filter Bank and precise temporal analysis. The frequency is defined from differentiation of the phase function without the limitation of the uncertainty principle, rather than integral transform in Fourier type of analysis. Most importantly, Fourier analysis would fail the sparse principle necessary to produce high fidelity sound by each component, ideal for Cochlear Implants.

In this invention, all sound signals will be represented by their sparse Intrinsic Mode Functions (IMF's). And the correct sound would be based on the instantaneous frequency and energy intensity at any given time. Before getting into the detailed implementation of the present invention, we will present the crucial difference of the present invention to the existing Fourier Filter Bank based Cochlear Implant systems. The key of the present invention is Empirical Mode Decomposition. The difference is that in Fourier analysis:

$$x ( t ) = {\sum }_{j = 1}^{N} {a}_{j} {e}^{i {ω}_{j} t} , \qquad ( 1 )$$

in which the amplitude, aⱼ, and frequency, ωⱼ, are all constants, we will use the adaptive Empirical Mode Decomposition (EMD). The same data x(t) is expanded in terms of the Intrinsic Mode Function (IMF), cⱼ(t), as

$$x ( t ) = {\sum }_{j = 1}^{N} {c}_{j} ( t ) = {\sum }_{j = 1}^{N} {a}_{j} ( t ) cos {θ}_{j} ( t ) = R {\sum }_{j = 1}^{N} {a}_{j} ( t ) {e}^{i {\int }_{t} {ω}_{j} ( τ ) d τ} , \qquad ( 2 )$$

where the frequency, ωⱼ(t), is defined as the time derivative of the adaptively determined phase function θⱼ(t); therefore, the transform from temporal space to frequency space is no longer through integration, but from differentiation; consequently, the frequency is no longer an average value in the time integration domain, but the instantaneous value. Of critical importance here for Cochlear Implants is the amplitude function aⱼ(t), which gives the natural modulation pattern (or sometimes envelope) automatically.

The differences between Fourier and EMD expansion are critical.

1. Since Fourier expansion is linear; it is very ineffective and requires a large number of terms to represent a given signal. For a signal with N data points, Fourier expansion requires N/2 terms. The same data expansion by EMD requires a maximum of log₂N terms. Many of the terms in Fourier transform are consisted of all harmonics, which are required for completeness, but they are artificial and should not be treated as natural sound signals.

2. The lack of harmonics of the sparse IMF representation is exactly what Cochlear Implants are calling for. Here, we can see the differences: without the benefit of cross component cancellation, the harmonics would generate noises. This is one of the leading reasons why Cochlear Implants would receive noisy sounds. For music, the harmonics are much richer. That is why the CI users would hear near cacophonous sound instead of beautiful music melody.

3. Most critically, none of the Fourier components could be free of interference from other sound sources when the sounds are nonlinear due to the shared harmonics. And all sounds are indeed nonlinear as indicated by the ubiquitous harmonics, which would mix hopelessly together.

According to the above detailed description of sound signal analysis and cochlear implants, in the present invention, we analyze sound signals based on HHT, which can improve the performance of cochlear implant in multiple sound source environments, and even realize the appreciation of music works.

In order to achieve the above-mentioned object of the invention, the present invention provides a speech processing method in a cochlear implant, which includes the following steps: obtaining a sound signal, and converting the sound signal into a digital signal; decomposing the digital signal using a mode decomposition method, obtaining a plurality of intrinsic mode functions (IMFs), and converting the plurality of intrinsic mode functions into instantaneous frequencies and instantaneous amplitudes or instantaneous energy intensities (squared amplitudes); sorting the instantaneous frequencies to corresponding the preset frequency bands of the electrodes in the cochlear implant; selecting N most energetic components from the corresponding frequency bands of the electrodes, and generating corresponding electrode stimulation signals according to the selected components. The advantage of this solution is that the frequency used in this solution is instantaneous frequency, so it is not limited by the uncertainty principle. In addition, in the speech processing method in a cochlear implant of the present invention, the digital signal is decomposed by the mode decomposition method, and harmonics will not be generated. Each electric signal represents the true neural signal of the sound, so even if it is superimposed, it will not generate unnecessary noise.

Preferably, the mode decomposition method includes Empirical Mode Decomposition method, Ensemble Empirical Mode Decomposition method, or Conjugate Adaptive Dyadic Masking Empirical Mode Decomposition method.

Preferably, the method further includes: before decomposing the digital signal using the mode decomposition method, using one of the following methods to suppress noise: adaptive filter bank method or artificial intelligence method.

Preferably, the method further includes: before decomposing the digital signal using the mode decomposition method, using one of the following methods to eliminate the cocktail party problem: Computational Auditory Scene Analysis, Non-negative Matrix Factorization, generative model modeling, beamforming, multi-channel blind source separation, Deep Clustering, Deep Attractor Network, and Permutation Invariant Training.

Preferably, the method further includes: selecting N most energetic components from the corresponding electrode frequency bands, wherein N≤6, and the energy values of these electrode frequency components are higher than the preset threshold. Here, the energy values of the electrode frequency bands are limited, mainly to prevent unnecessary noise from being generated at the speech pause.

Preferably, correcting the selected intrinsic mode functions includes automatic gain control, which adjusts the stimulation signal of each electrode according to patient's audiogram, if residual hearing capability remains.

Preferably, the method further includes: generating the electrode stimulation signal corresponding to the selected intrinsic mode functions by one of the following methods: Simultaneous Analog Signal, Compression Analysis, and Continuous Interleaved Sampling.

Preferably, the preset electrode frequency bands in the cochlear implant correspond to the electrodes in the cochlear implant one to one, and the number of electrodes is greater than or equal to 20 at the present time, and this number could increase when technology warrants. In the present invention, as the number of electrodes in the cochlear implant increases, the set of instantaneous frequencies can be increased accordingly, and the increase in the number of electrodes can make the sound generated by the electrodes more realistic.

In order to reduce signal processing time and cost, the present invention also provides another cochlear implant speech processing method, which includes the following steps: obtaining a sound signal, and converting the sound signal into a digital signal; decomposing the digital signal using an adaptive filter bank method, obtaining a plurality of pseudo-intrinsic mode functions, and converting the plurality of pseudo-intrinsic mode functions into instantaneous frequencies and instantaneous amplitudes or instantaneous energy intensities; sorting the instantaneous frequencies to corresponding the preset frequency bands of electrodes in the cochlear implant; selecting N most energetic components from the corresponding frequency bands of the electrodes, and generating corresponding electrode stimulation signals according to the selected components. Using the adaptive filter bank method for signal decomposition can effectively increase the speed of signal processing and reduce computation costs.

Preferably, the adaptive filter bank is a running mean filter bank or a median filter bank.

In another aspect of the invention, there is provided a speech processing system in a cochlear implant, which includes a sound receiving module, a sound processing module, and a signal transmission module, wherein the sound receiving module is configured to receive a sound signal, and convert the sound signal into a digital signal; the sound processing module is configured to perform the following operations: processing the digital signal to obtain a plurality of intrinsic mode functions or pseudo-intrinsic mode functions, and converting the plurality of intrinsic mode functions or pseudo-intrinsic mode functions into instantaneous frequencies and instantaneous amplitudes or instantaneous energy intensities; sorting the instantaneous frequencies to corresponding the preset frequency bands of the electrodes in the cochlear implant; selecting N most energetic components from the corresponding frequency bands of the electrodes, and generating corresponding electrode stimulation signals according to the selected components; and the signal transmission module is configured to transmit the electrode stimulation signals generated by the sound processing module to the electrodes in the cochlear implant, so that the electrodes generate stimulation signals corresponding to the sound. Preferably, the speech processing system described above operates mostly in time domain; and based on the decomposition method, the signals for each electrode are in terms of instantaneous frequencies and instantaneous energy intensities as a function of time without the help of spectral representation in any form.

There has always been a misunderstanding of sound, and it is believed that all sound signals can be decomposed into sine waves, that is, sounds are represented by Fourier frequencies. The invention overcomes the false knowledge in sound analysis, and analyzes sound signals in the time domain based on Hilbert-Huang transform. In the cochlear implant speech processing method and system of the present invention, sound signals are analyzed in the time domain, and frequencies used are instantaneous frequencies, which is not limited by the uncertainty principle. In addition, in the present invention, each electric signal represents the true neural signal of the sound, and harmonics will not be generated, so there is no unnecessary noise.

### BRIEF DESCRIPTION OF THE DRAWINGS

FIG. 1 is the flow chart of the cochlear implant speech processing method of the present invention.

FIG. 2 is the sound signal diagram of the Chinese sentence, ‘Zeng xiansheng zao’ (which means ‘Good Morning Mr. Zeng’).

FIG. 3 is the sound components diagram of the sound signals in FIG. 2 after being filtered by a Fourier bandpass filter bank.

FIG. 4 is the Fourier time-frequency diagram of the sound signals in FIG. 2.

FIG. 5 is the sound components diagram of the sound signals in FIG. 2 after EMD decomposition.

FIG. 6 is the Hilbert time-frequency diagram of the sound signals in FIG. 2.

FIG. 7 is the IMF components diagram of the sound signals in FIG. 2 obtained by Ensemble Empirical Mode Decomposition, in which the noise level is low (1%), and there are only 2 members in the ensemble.

FIG. 8 is the IMF components diagram of the sound signals in FIG. 2 obtained by Ensemble Empirical Mode Decomposition, in which the noise level is high (10%), and there are 16 members in the ensemble.

FIG. 9 is the time-frequency diagram of the 20-electrode frequency band simulation of the IMFs given in FIG. 5, but the frequency axis is plotted in logarithmic scale.

FIG. 10 is the time-frequency diagram of the 20-electrode frequency band simulation of the IMFs given in FIG. 7 but the frequency axis is plotted in logarithmic scale.

FIG. 11 is the time-frequency diagram of the 20-electrode frequency band simulation of the IMFs given in FIG. 8 but the frequency axis is plotted in logarithmic scale.

FIG. 12 shows the cochlear implant speech processing system of the present invention.

### DETAILED DESCRIPTION OF THE INVENTION

In the following, with the reference to the accompanying drawings and the preferred embodiments of the present invention, the technical means adopted by the present invention to achieve the intended purpose of the present invention will be further explained.

### EXAMPLE 1

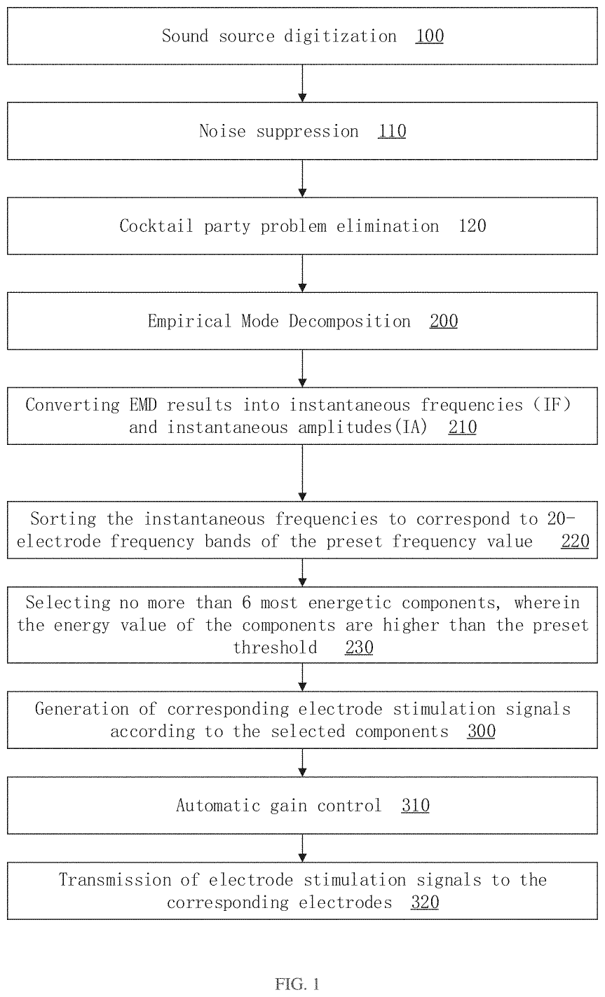

Referring to FIG. 1, it is detailed implementation of the cochlear implant speech processing method of the present invention. In step 100, a sound signal is digitized, wherein the sampling frequency can be selected as required. To achieve higher fidelity, a high-frequency sampling frequency, 22 KHz or 44 KHz can be used (22 KHz and 44 KHz are the sampling frequencies used by current mainstream acquisition cards). Because some noise may appear in sound, the noise needs to be suppressed or eliminated, in step 110, noise suppression is performed. In noise suppression, adaptive filters can be used, or artificial intelligence methods, such as RNN, DNN, MLP, etc., can be used. In addition, the “cocktail party problem” is also an important issue in the field of speech recognition. The current speech recognition technology can already recognize a person's words with high accuracy, but when there are two or more people speaking, the speech recognition rate will be greatly reduced. This problem is called the cocktail party problem. In step 120, the following techniques can be used to eliminate the cocktail party problem: for single-channel situations, Computational Auditory Scene Analysis (CASA), Non-negative Matrix Factorization (NMF) and generative model modeling can be used; for multi-channel situations, technologies such as beamforming or multi-channel blind source separation can be used; some techniques based on deep learning can also be used, such as Deep Clustering, Deep Attractor Network (DANet) and Permutation Invariant Training (PIT).

In step 200, the signal after the noise filtering is decomposed by a mode decomposition method to obtain the Intrinsic Mode Functions (IMFs) of the sound signal. The mode decomposition method refers to any mode decomposition method that can obtain the Intrinsic Mode Function components (IMFs) of the signal. The mode decomposition method includes Empirical Mode Decomposition (EMD), Ensemble Empirical Mode Decomposition (EEMD), Conjugate Adaptive Dyadic Masking Empirical Mode Decomposition (CADM-EMD). In step 210, the mode decomposition result is converted into Instantaneous Frequencies (IF) and Instantaneous Amplitudes (IA) or instantaneous energy intensities. In step 220, according to the instantaneous frequency values, the Intrinsic Mode Function components are assigned to the frequency bands corresponding to the electrodes. The number of electrodes and the frequency bands corresponding to the electrodes have been preset. The greater the number of electrodes, the stronger the frequency resolution capability, and the better the effect achieved. However, there may be problems such as crosstalk between multiple electrodes, and the length of the implant is limited, so the number of electrodes that can be accommodated is also limited. Therefore, the number of electrodes should be appropriate. The frequencies corresponding to the electrodes should be determined according to the characteristics of the sound. For frequency bands where the sound frequencies are more concentrated (such as lower than 1000 Hz), the electrodes can be set densely to improve the frequency resolution. For frequency bands where the sound frequencies are not concentrated (such as higher than 1000 Hz), the number of electrodes can be set less, on an approximately logarithmic scale. To follow the principle of a limited number of electrodes, the number of electrodes can be selected as 20, for example, and the frequency values we specify are: 80, 100, 128, 160, 200, 256, 320, 400, 512, 640, 800, 1024, 1280, 1600, 2048, 2560, 3200, 4096, 5120, 6400, 8192. The specified 21 frequency values define 20 frequency bands, and every two adjacent frequencies define one frequency band. The first frequency band is 80-100 Hz, the second frequency band is 100-128 Hz, . . . , the 20th frequency band is 6400-8192 Hz. These 20 frequency bands correspond to the electrodes in the cochlear implant, and each electrode corresponds to a frequency band. It can be found from the above frequency values that a scale contains 3 frequencies, which are used to distinguish different frequencies in the same scale. In the present invention, more electrodes will improve the frequency difference, thereby improving the final sound quality. For example, the high cut-off frequency and low cut-off frequency can be changed, and up to 25 electrodes can be deployed in a small total range, and a better frequency difference between electrodes can be achieved. When the number of electrodes is 25, for example, the corresponding frequencies can be as follows: 50, 64, 75, 90, 105, 128, 150, 180, 210, 256, 300, 360, 420, 512, 600, 720, 840, 1024, 1200, 1440, 1680, 2048, 2400, 2880, 3360, 4096. Like the case of 20 electrodes, each electrode corresponds to a frequency band. The frequency band corresponding to the first electrode is 50-64 Hz, the frequency band corresponding to the second electrode is 64-75 Hz, . . . , the frequency band corresponding to the twenty-fifth electrode is 3360-4096 Hz. As the number of electrodes increases, the cochlear implant using the speech processing method of the present invention will gain higher and higher frequency resolution capabilities. Because when the number of electrodes increases, the instantaneous frequency set can be increased accordingly, and the resolution of the electrodes to the sound is improved, so the sound produced by the electrodes will be more realistic. Therefore, when using 88 electrodes, for example, we should be able to fully enjoy piano music, albeit with less colorful (timbre) of sounds because the piano sound for each key is highly nonlinear. After pairing the Intrinsic Mode Function components to the corresponding electrode frequency bands, then, in step 230, the most energetic components are selected from the corresponding electrode frequency bands, the number of selected electrodes is not more than 6 at the present time, and the number could increase when technology warrants, and the energy values of these components are higher than preset threshold. Because when multiple electrodes are stimulated at the same time, crosstalk may occur between the electrodes, so current experiments show that when the number of selected electrodes is not more than 6, the influence between the electrodes is small. In addition, the purpose of threshold setting here is that in speech, because there are pauses between different words, phrases or sentences, electrode stimulation is not needed during the pause, and the energy value of the sound component is low at this time, thus the threshold is used to filter the weak energy components at the pause. The threshold can be selected between 10% and 20% of the average energy of the sound.

In step 300, corresponding electrode stimulation signals are generated according to the selected components. The following methods can be used to generate electrode signals: Simultaneous Analog Signal (SAS), Compressive Analysis (CA), Continuous Interleaved Sampling (CIS). In step 310, automatic gain control is performed to limit its loudness. The automatic gain control is mainly based on the audiogram of the hearing-impaired patient to obtain the sound perception ability of the patient in different frequency ranges, and then adjust the stimulation signal of electrode corresponding to each frequency according to the patient's hearing test results. This step is optional, only for patients who still have remaining hearing ability. Then, in step 320, the electrode stimulation signal is transmitted to the corresponding electrode. When generating electrode signals, although there are some other methods that also claim to use selective frequency bands, such as Advanced Combinatorial Encoders (ACE), Dynamic Peak Picking, Spectral Peak (SPEAK), Current Steering, etc., but it should be noted that the effects of these methods are not obvious, because the implementation of these methods is based on the Fourier filter bank, which is always affected by virtual harmonics. When transmitted to a limited number of electrodes, any electric signal must represent the real neural signal of the sound, but the harmonic signal is not a real sound signal. In hearing aids, the cancellation and combination of harmonics will cause the fundamental wave to be amplified, resulting in louder annoying and yet unclear sound. In the cochlear implants, the harmonics are recetified, and they cannot be eliminated by combination and cancellation, which will cause unnecessary noise. Therefore, if the sound is full of harmonics (such as the sound in an instrument), the problem will become worse. These harmonics will all be intertwined and become inseparable, making music appreciation impossible.

Compared with the cochlear implant speech processing method based on the Fourier principle, the present invention has the advantages of: (1) The frequencies in the present invention are instantaneous frequencies, so it is not limited by the uncertainty principle; while the Fourier transform is an integral transform, and any method based on integral transform cannot obtain instantaneous frequencies; (2) In the HHT based cochlear implant speech processing method of the present invention, no harmonics will be generated, and each electric signal represents the true neural signal of the sound; while for Fourier based cochlear implants, there are some harmonics in the signal, which cannot be eliminated, resulting in a lot of unnecessary noise; (3) In the present invention, a larger number of electrodes can be used to improve the frequency difference, thereby improving the final sound quality; but cochlear implants based on the Fourier principle have harmonics, even if the number of electrodes is increased, the harmonics cannot be eliminated by combination and cancellation, that is, the final sound quality cannot be improved by increasing the number of electrodes; and (4) In the present invention, the sound components can be selectively amplified according to the patient's hearing test results, to preserve the natural cochlear function of some hearing-impaired patients.

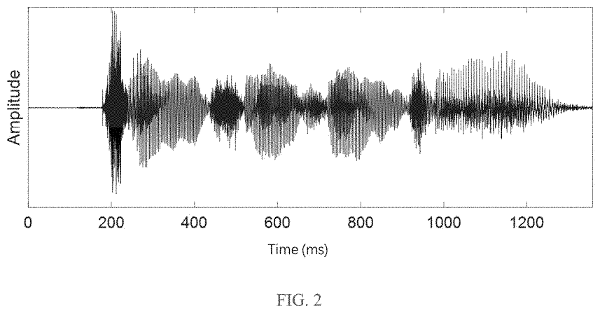

FIG. 2 shows the speech signal data of the Chinese sentence, ‘Zeng xiansheng zao’.

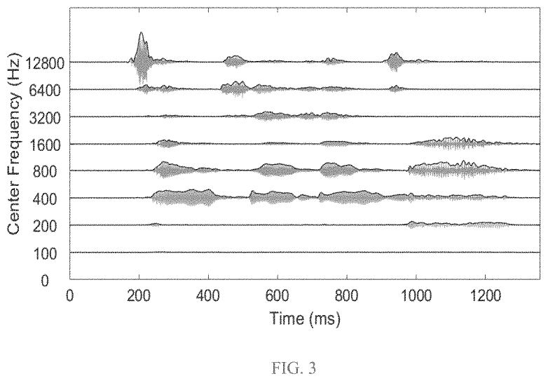

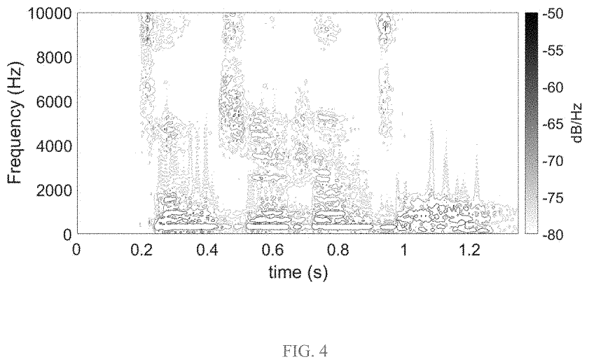

FIG. 3 is the sound components diagram of the sound signals in FIG. 2 after being filtered by a Fourier bandpass filter bank. FIG. 3 shows the seven band-pass filter frequency bands used in a typical cochlear implant at present, and the result of the Fourier band-pass filter of 8 components will be given. The envelope of these sound components will be the input of the cochlear implant electrodes. FIG. 4 is a detailed enlarged view of the Fourier time-frequency spectrum of the Chinese sentence ‘Zeng xiansheng zao’ in FIG. 2, and it vividly shows the regularity of harmonics. These harmonics are necessary for the representation of nonlinear signal integrity, but they are not truly natural sounds. When they are superimposed, a non-linear distortion waveform will be produced. However, for cochlear implants that use sound signal component envelopes, harmonics will no longer be superimposed to form the fundamentals, rather harmful noise will be generated at the corresponding frequency.

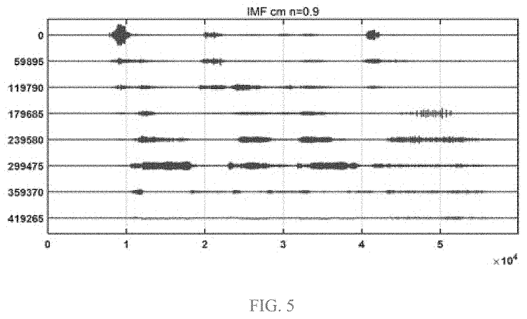

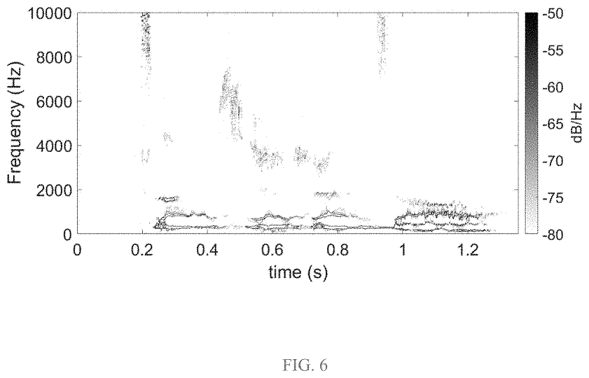

FIG. 5 is the 8 frequency bands generated by the filter bank of the sound signals in FIG. 2 after EMD decomposition. FIG. 5 seems to be similar to the filtered result of the band-pass filter bank in FIG. 3, but, as discussed above, the filtered result of the band-pass filter bank itself does not represent the sound well. FIG. 6 is the Hilbert time-frequency spectrum of the Chinese sentence “Zeng xiansheng zao” in FIG. 2, covering a frequency range of 0-10000 Hz. Among them, the energy concentration around 300 Hz represents the vibration of the vocal cords, the main energy concentration between 400-1000 Hz represents the resonance of the articulator, and the high-frequency energy between 2000-5000 Hz represents the reflection of the vocal tract. These frequency ranges depend on the person's mouth shape and size and vary from person to person. These frequencies increase the intensity of the sound. It can be seen from FIG. 6 that only few energy values exceed 1000 Hz. More importantly, there are no harmonics in these high-frequency energies, and the time and frequency values are not limited by the uncertainty principle.

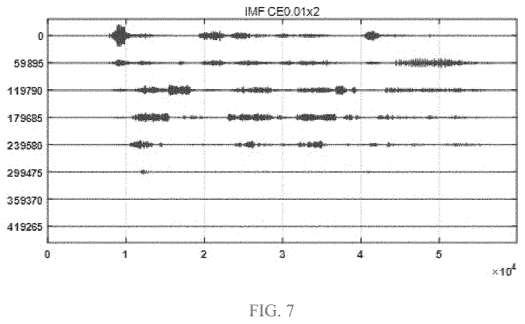

FIG. 7 is the IMF components diagram obtained by Ensemble Empirical Mode Decomposition, in which the noise level is low (1%), and there are only 2 members in the ensemble. Comparing the IMF components in FIG. 7 and FIG. 5, it can be seen that there is a big difference between the two. Ensemble Empirical Mode Decomposition (EEMD) is a noise-assisted data analysis method for the deficiencies of the Empirical Mode Decomposition (EMD) method. EEMD will effectively solve the frequency mixing phenomenon in EMD.

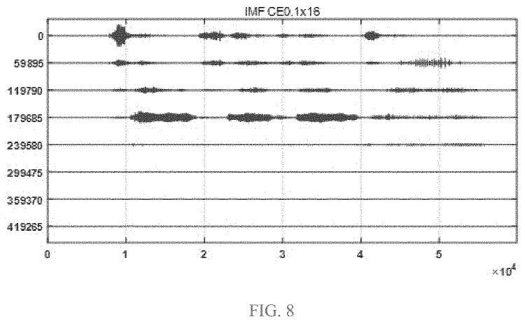

FIG. 8 is the IMF components diagram obtained by Ensemble Empirical Mode Decomposition, in which the noise level is high (10%), and there are 16 members in the ensemble. Comparing the IMF components in FIG. 8 with FIG. 7 and FIG. 5, it can be seen that the IMF components in FIG. 8 are very different from the IMF components in FIG. 5 or FIG. 7.

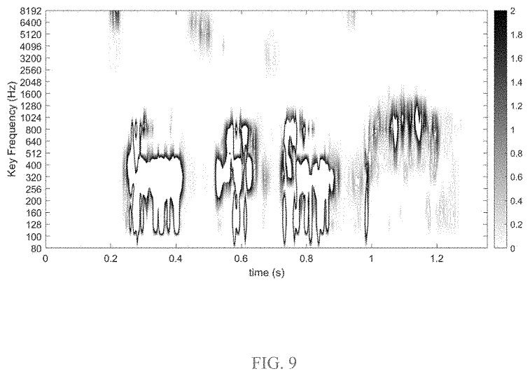

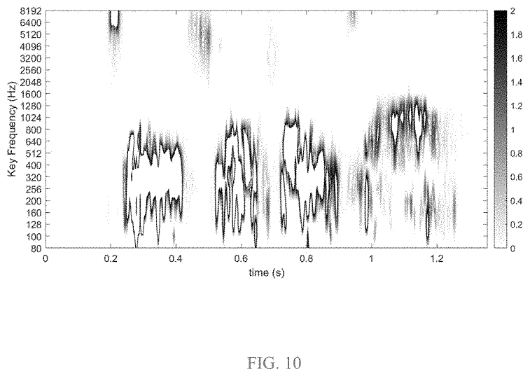

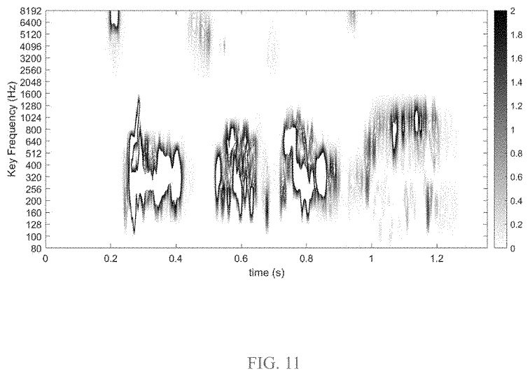

FIG. 9 is the time-frequency diagram of the 20-electrode frequency band simulation of the IMFs given in FIGS. 5 and 6, because of the near logarithmic distribution of our ears. In FIG. 9, the frequency axis is in logarithmic scale, which is also true for FIGS. 10 and 11. The frequencies corresponding to the 20 electrodes are: 80, 100, 128, 160, 200, 256, 320, 400, 512, 640, 800, 1024, 1280, 1600, 2048, 2560, 3200, 4096, 5120, 6400, 8192. Comparing the Hilbert time-frequency diagrams in FIG. 9 with FIG. 6, FIG. 9 provides more details than shown in FIG. 6. It is similar in quality to the full resolution spectrum in FIG. 6, and can contain many fine frequency features of speech.

FIG. 10 is the time-frequency diagram of the 20-electrode frequency band simulation of the IMFs given in FIG. 7. The frequencies corresponding to the electrodes are the same as in FIG. 9. Again, FIG. 10 provides more details than shown in FIG. 6, it is similar in quality to the spectrum given in FIG. 9.

FIG. 11 is the time-frequency diagram of the 20-electrode frequency band simulation of the IMFs given in FIG. 8. The frequencies corresponding to the electrodes are the same as in FIG. 9. Again, FIG. 11 provides more details than shown in FIG. 6, it is similar in quality to the spectrum given in FIG. 9.

FIG. 5, FIG. 7 and FIG. 8 respectively decompose the sound signals in FIG. 2 using different mode decomposition methods, and obtain the corresponding IMF components after decomposition by different methods. It can be seen from the figures that the IMF components decomposed by different methods are very different, and the envelopes of the corresponding IMF components are also very different. However, after being converted into instantaneous frequencies and instantaneous amplitudes or instantaneous energy intensities (squared amplitudes), the time-frequency diagrams are similar, and the electrode stimulation signals of cochlear implant are related to frequencies and energies. Therefore, different decomposition methods will produce basically the same electrode stimulation signals.

### EXAMPLE 2

Furthermore, in order to save time, any method similar or equivalent to EMD can be used to replace EMD. For example, the running mean or median method of successive running different window sizes is repeatedly applied as needed, as high-pass filtering or other time-domain filtering for filtering the input signals. For example, in the running mean method, there is no guarantee that the signal obtained is a real IMF, which is a requirement for generating accurate and meaningful instantaneous frequencies. However, since spectrum analysis is not used, the approximate value is acceptable. Taking the successive running mean as an example, the steps should be as follows. First, the data is decomposed by successive running mean:

$$x ( t ) - {〈 x ( t ) 〉}_{n 1} = {h}_{1} ( t ) , \\ {〈 x ( t ) 〉}_{n 1} - {〈 {〈 x ( t ) 〉}_{n 1} 〉}_{n 2} = {h}_{2} ( t ) , \\ {〈 {〈 x ( t ) 〉}_{n 1} 〉}_{n 2} - {〈 {〈 {〈 x ( t ) 〉}_{n 1} 〉}_{n 2} 〉}_{n 3} = {h}_{3} ( t ) , \\ … \\ {〈 {〈 {〈 {〈 x ( t ) 〉}_{n 1} 〉}_{n 2} 〉}_{n 3} … 〉}_{N - 1} - {〈 {〈 {〈 {〈 x ( t ) 〉}_{n 1} 〉}_{n 2} 〉}_{n 3} … 〉}_{N} = {h}_{N} ( t ) \\ … \\ x ( t ) = {\sum }_{j = 1}^{N} {h}_{j} ( t ) + {〈 {〈 {〈 {〈 x ( t ) 〉}_{n 1} 〉}_{n 2} 〉}_{n 3} … 〉}_{N} \qquad ( 3 )$$

in which <F>ₙⱼ represents the running mean of the window size nj (or the running median, reused if necessary). The advantage of using a rectangular filter is that the filter is adaptive, and the response function of the rectangular filter is well known. In addition, the repeated use of the rectangular filter actually changes the known response function of the filter. Repeating twice will produce a triangular filter, and repeating more than four times will produce a response function close to the Gaussian shape. The key parameter for using this filter is the size of the window. According to formula (3), we draw the following conclusions, if the sampling frequency is 22050 Hz, the rectangular filter and EMD have the following equivalent relationship:

nj=3˜7,000 Hz (4)

nj=7˜3,500 Hz

nj=15˜1,500 Hz

nj=31 ˜700 Hz

nj=61 ˜350 Hz

nj=121˜180 Hz

nj=241˜90 Hz

nj=481˜45 Hz

There is no need to continue filtering, because in any case we cannot hear sounds with frequencies lower than the next filter step. The disadvantage of using filters is that no filter is clearer than the above-mentioned EMD.

Selective enlargement or reduction can be realized as in formula (3), and the reconstructed signal y(t) is obtained as:

$$y ( t ) = {\sum }_{j = 1}^{N} {a}_{j} \times {h}_{j} ( t ) + {〈 {〈 {〈 {〈 x ( t ) 〉}_{n 1} 〉}_{n 2} 〉}_{n 3} … 〉}_{N} \qquad ( 5 )$$

in which the value of aⱼ can be determined according to patient's audiogram.

Because EMD is more time-consuming, but even so, its computational complexity can still be comparable to Fourier transform. If the filter method is used, the sound may not be particularly clear, because the mean filter does spread the filtered result in a wider time domain. The final result will not be as clear as the complete EMD method, but the filter method can be simpler and cheaper to implement.

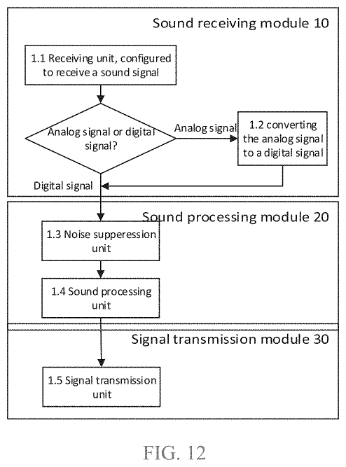

Referring to FIG. 12, it shows a cochlear implant speech processing system according to an embodiment of the present invention. The speech processing system includes a sound receiving module 10, a sound processing module 20 and a signal transmission module 30. The sound receiving module 10 is configured to receive a sound signal, and convert the sound signal into a digital signal. The sound processing module 20 is configured to perform the following operations: reducing the noise of the received sound digital signal, decomposing the sound signal, and converting the decomposed signal components into instantaneous frequencies and instantaneous amplitudes or instantaneous energy intensities; corresponding the instantaneous frequencies to the electrode frequency bands; selecting several most energetic frequency bands; and generating electrode stimulation signals corresponding to the frequency bands with the highest energy intensity. The principles and detailed steps involved in the key parts of the sound processing module are the same as those listed in the cochlear implant speech processing method. After the sound processing module 20 receives the digital sound signal, a noise reduction unit performs noise suppression on the sound signal and eliminates the cocktail party problem. Then, a sound processing unit will process the sound signal through an adaptive filter bank to obtain a plurality of intrinsic mode functions or pseudo-intrinsic mode functions. Among them, the adaptive filter bank includes mode decomposition filter bank, mean filter bank. The mode decomposition filter bank adopts any method in the present invention that can obtain IMF components, such as Empirical Mode Decomposition (EMD), Ensemble Empirical Mode Decomposition (EEMD), or Conjugate Adaptive Dyadic Masking Empirical Mode Decomposition (CADM-EMD). In addition to the above-mentioned various empirical mode decomposition methods and improved signal decomposition methods based on them, an adaptive filter bank such as mean filter bank can also be used to obtain pseudo-IMFs. The IMFs or pseudo-IMFs obtained by the adaptive filter bank are converted into instantaneous frequencies and instantaneous amplitudes or instantaneous energy intensities. The obtained instantaneous frequencies are corresponded to the electrode frequency bands of the preset frequency value, at most 6 most energetic components are selected from the corresponding electrode frequency bands, and the energies in these frequency bands are greater than the preset threshold value. Then, the corresponding electrode stimulation signals are generated according to the selected components, and the loudness of each signal component is controlled through automatic gain control. When performing automatic gain control on the signal, it is possible to control the amplification of each frequency component according to the patient's audiogram, which can preserve patient's natural cochlear function. The signal transmission module 30 is configured to transmit the electrode stimulation signals generated by the sound processing module to the electrodes in the cochlear implant, so that the electrodes can correctly generate stimulation signals corresponding to the sound in real time.

The above are only the preferred embodiments of the present invention, and do not limit the present invention in any form. Although the present invention has been disclosed as above in preferred embodiments, it is not intended to limit the present invention. Anyone who is familiar with the field, without departing from the scope of the technical solution of the present invention, can use the technical content disclosed above to make slight changes or modifications into equivalent embodiments with equivalent changes. Any simple modifications, equivalent changes and variations made to the above embodiments based on the technical essence of the present invention without departing from the technical solution of the present invention still fall within the scope of the technical solution of the present invention.

1. A speech processing method in a cochlear implant, characterized in that, it includes the following steps:
obtaining a sound signal, and converting the sound signal into a digital signal;
decomposing the digital signal using a mode decomposition method, obtaining a plurality of intrinsic mode functions, and converting the plurality of intrinsic mode functions into instantaneous frequencies and instantaneous amplitudes or instantaneous energy intensities;
sorting the instantaneous frequencies to corresponding the preset frequency bands of the electrodes in the cochlear implant;
selecting N most energetic components from the corresponding frequency bands of the electrodes, and generating corresponding electrode stimulation signals according to the selected components.

2. The speech processing method of claim 1, characterized in that, the mode decomposition method includes Empirical Mode Decomposition method, Ensemble Empirical Mode Decomposition method, or Conjugate Adaptive Dyadic Masking Empirical Mode Decomposition method.

3. The speech processing method of claim 1, characterized in that, it further includes:
before decomposing the digital signal using the mode decomposition method, using one of the following methods to suppress noise: adaptive filter bank method or artificial intelligence method.

4. The speech processing method of claim 1, characterized in that, it further includes:
before decomposing the digital signal using the mode decomposition method, using one of the following methods to eliminate the cocktail party problem: Computational Auditory Scene Analysis, Non-negative Matrix Factorization, generative model modeling, beamforming, multi-channel blind source separation, Deep Clustering, Deep Attractor Network, and Permutation Invariant Training.

5. The speech processing method of claim 1, characterized in that, it further includes:
selecting N most energetic components from the corresponding electrode frequency bands, wherein N≤6, and the energy values of these electrode frequency components are higher than the preset threshold.

6. The speech processing method of claim 1, characterized in that, it further includes:
automatic gain control, which adjusts the stimulation signal of each electrode according to patient's audiogram.

7. The speech processing method of claim 1, characterized in that, it further includes:
generating the electrode stimulation signal corresponding to the selected intrinsic mode functions by one of the following methods: Simultaneous Analog Signal, Compression Analysis, and Continuous Interleaved Sampling.

8. The speech processing method of claim 1, characterized in that, it further includes:
the preset frequency bands in the cochlear implant correspond to the electrodes in the cochlear implant one to one, and the number of electrodes is greater than or equal to 20.

9. A speech processing method in a cochlear implant, characterized in that, it includes the following steps:
obtaining a sound signal, and converting the sound signal into a digital signal;
decomposing the digital signal using an adaptive filter bank method, obtaining a plurality of pseudo-intrinsic mode functions, and converting the plurality of pseudo-intrinsic mode functions into instantaneous frequencies and instantaneous amplitudes or instantaneous energy intensities;
sorting the instantaneous frequencies to corresponding the preset frequency bands of electrodes in the cochlear implant;
selecting N most energetic components from the corresponding frequency bands of the electrodes, and generating corresponding electrode stimulation signals according to the selected components.

10. The speech processing method of claim 9, characterized in that, the adaptive filter bank is a mean filter bank or a median filter bank.

11. A speech processing system in a cochlear implant using the speech processing method of claim 1, characterized in that, the speech processing system includes a sound receiving module, a sound processing module, and a signal transmission module, wherein
the sound receiving module is configured to receive a sound signal, and convert the sound signal into a digital signal;
the sound processing module is configured to perform the following operations:
processing the digital signal to obtain a plurality of intrinsic mode functions or pseudo-intrinsic mode functions, and converting the plurality of intrinsic mode functions or pseudo-intrinsic mode functions into instantaneous frequencies and instantaneous amplitudes or instantaneous energy intensities; sorting the instantaneous frequencies to corresponding the preset frequency bands of the electrodes in the cochlear implant;
selecting N most energetic components from the corresponding frequency bands of the electrodes, and generating corresponding electrode stimulation signals according to the selected components; and
the signal transmission module is configured to transmit the electrode stimulation signals generated by the sound processing module to the electrodes in the cochlear implant, so that the electrodes generate stimulation signals corresponding to the sound.

12. A speech processing system in a cochlear implant using the speech processing method of claim 9, characterized in that, the speech processing system includes a sound receiving module, a sound processing module, and a signal transmission module, wherein
the sound receiving module is configured to receive a sound signal, and convert the sound signal into a digital signal;
the sound processing module is configured to perform the following operations:
processing the digital signal to obtain a plurality of intrinsic mode functions or pseudo-intrinsic mode functions, and converting the plurality of intrinsic mode functions or pseudo-intrinsic mode functions into instantaneous frequencies and instantaneous amplitudes or instantaneous energy intensities; sorting the instantaneous frequencies to corresponding the preset frequency bands of the electrodes in the cochlear implant;
selecting N most energetic components from the corresponding frequency bands of the electrodes, and generating corresponding electrode stimulation signals according to the selected components; and
the signal transmission module is configured to transmit the electrode stimulation signals generated by the sound processing module to the electrodes in the cochlear implant, so that the electrodes generate stimulation signals corresponding to the sound.

13. The speech processing system of claim 11, characterized in that, it operates mostly in time domain; and based on the decomposition method, the signals for each electrode are in terms of instantaneous frequencies and instantaneous energy intensities as a function of time without the help of spectral representation in any form.

14. The speech processing system of claim 12, characterized in that, it operates mostly in time domain; and based on the decomposition method, the signals for each electrode are in terms of instantaneous frequencies and instantaneous energy intensities as a function of time without the help of spectral representation in any form.
